(19) United States
(12) Patent Application Publication
(10) Pub. No.: US 2007/0258917 A1
(43) Pub. Date: Nov. 8, 2007

(54) Process for removing or lightening stains caused by skin coloring agents

(75) Inventors: Isabelle Hansenne, Westfield, NJ (US); Paula Reis, Eatontown, NJ (US); Angelike Galdi, Westfield, NJ (US)

Correspondence Address: L'OREAL USA/ PATENT DEPARTMENT, 30 TERMINAL AVENUE, CLARK, NJ, 07066, US

(73) Assignee: L'OREAL, Paris (FR)

(21) Appl. No.: 11/542,496

(22) Filed: Oct. 3, 2006

Related U.S. Application Data
(60) Provisional application No. 60/797,551, filed on May 3, 2006.

(51) Int. Cl. A61K 8/19

(52) U.S. Cl. 424/59; 424/62

(57) ABSTRACT

A process for removing or lightening a stain from human skin caused by a sunless tanning product involving contacting the stain with a stain removing composition containing at least one skin-abrading metal oxide to form a clean or lightened skin surface.

### BACKGROUND OF THE INVENTION

More consumers are becoming aware of the skin damaging effects of ultra-violet radiation from repeated sun exposure, causing “self-tanning” or “sunless tanning” products to gain popularity in recent years. Most such products contain the active ingredient dihydroxyacetone (DHA), a skin coloring agent which reacts with protein in skin's stratum corneum to produce a brown color somewhat similar to the color of suntan.

One major problem associated with DHA-based self tanning products is the difficulty of even application. An aqueous solution of DHA is colorless and its reaction with skin to produce brown color is quite slow. Since it takes several hours for the color to become visible and about 24 hours to complete color development, it is difficult for the user to know if the solution has been correctly applied to the desired areas of the body or if the application is even.

To minimize this problem, many DHA-based self-tanning products now contain auxiliary skin coloring agents to assist the users in identifying areas of uneven or unwanted application. The auxiliary skin coloring agents may consist of any combination of food colors, organic dyes or inorganic pigments. However, the auxiliary skin coloring agents can have the unintended effects of staining areas which should not “tan,” such as the palms or fingernails. More important, the auxiliary skin coloring agents can indicate those areas that will be affected by the DHA over time. In this way, the user can quickly spot areas from which the DHA should be removed before any reaction can occur.

Regardless of which type of skin coloring agent is used the subsequent color stain formed on the skin can be very difficult to remove or lighten. Even after repeated washing with soap and water, complete removal or lightening may be difficult to achieve.

While other methods of stain removal exist such as the use of a strong detergent, i.e. sodium lauryl sulfate, or repeated scrubbing with, for example, a brush, these can be injurious to skin and/or cause severe drying thereof. As a result, there is a need for a “stain remover” that is safe, easy to use, and has the ability to remove or easily lighten the skin staining DHA and/or colorants found in sunless tanning products.

### BRIEF SUMMARY OF THE INVENTION

The present invention generally relates to a process for removing or lightening stains from human skin caused by sunless tanning products involving contacting the stain with a stain removing composition containing at least one skin-abrading metal oxide.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

The present invention is based on the surprising discovery that the use of a stain removing composition containing at least one metal oxide skin-abrading agent, on human skin stained by a sunless tanning product, effectively removes or lightens said stain.

Suitable skin-abrading metal oxide particles may include magnesium oxide and aluminum oxide. These particles have a purity of at least about 95%, preferably at least about 99%. Their average particle size preferably ranges from about 100 to about 180 μm. Aluminum oxide is preferred for use in the present invention, particularly in an anhydrous crystalline form (corundum).

Aluminum oxide particles are available as particles calcined at high temperature, until the α-Al₂O₃ crystalline structure of corundum is obtained, and then treated in order to form grains having sharp edges and having a given particle size distribution, the particles preferably having an average particle diameter of between about 100 and about 180 μm, more preferably between about 130 and about 150 μm. Their distribution is preferably such that none of the particles has a diameter greater than 250 μm. Such particles are available commercially from MarkeTech International under the trade name Dermagrain™. The particles referenced as Dermagrain™ 900 are composed of crystalline α-alumina with a purity of 99.55% having an average particle size diameter of approximately 140 μm, the particles all having a diameter of less than 250 μm. Less than 3% of the particles have a diameter of less than 105 μm. Other particles are commercially available from Industrial Supply under the trade names ARL 100 and ARL 120. They are aluminum oxide particles having an average particle diameter of about 120 and about 100 μm, respectively, and a particle size distribution ranging from about 75 to about 212 μm, and from about 63 to about 180 μm, respectively.

In another embodiment, the metal oxide particles used in the composition according to the invention may be magnesium oxide preferably having an average particle size ranging from about 100 to about 180 μm. An example of particles of this type is sold by MarkeTech International under the trade name Magnaderm™ 100. They are particles having an average particle diameter of approximately 120 μm and a purity of at least 99%.

The skin abrading metal oxide will typically be present in an amount sufficient to quickly and easily remove or lighten a stain located on the targeted area of skin. Preferably, the skin abrading metal oxide particles may be present in concentrations ranging from about 0.5 to about 40% by weight, preferably from about 10 to about 30% by weight, and more preferably from about 15 to about 25% by weight, all weights based on the total weight of the stain removing composition.

The composition preferably has a pH of greater than about 4 and less than about 8, preferably less than or equal to about 7, and more preferably between about 5.5 and about 7

Determining the precise amount of skin-abrading metal oxide to be used for a particular formulation is well within the skill of the ordinary artisan. In fact, because of the variation in skin types, it is preferable that formulations within the scope of this invention be available in different grades of abrasiveness (e.g., very fine, fine, and medium) so that the user can determine which is optimal for his (or her) own use.

The stain removing composition may be provided in the form of a lotion, gel, fluid or cream. It may comprise various adjuvants and advantageously may include at least one heterogeneous polysaccharide. This is because the Applicant has found that these compounds make it possible to reduce the discomfort associated with the use of the above-mentioned abrasive metal oxide particles. This heterogeneous polysaccharide can be an alginate or alternatively a heterogeneous polysaccharide comprising at least one fucose unit, comprising in particular fucose, galactose and galacturonic acid units, in particular a linear sequence of α-L-fucose, of α-D-galactose and of galacturonic acid. Such a polysaccharide is available in particular in the form of a 1% solution in water from Solabia under the trade name Fucogel 1000 PP®.

The stain removing composition may be applied to the stained skin by manual massaging with the fingertips or by mechanical massaging using a vibrating device provided with a massaging head equipped with a pad as disclosed, for example, in U.S. Pat. No. 6,652,888.

The at least one skin-abrading metal oxide may be present in any conventional cosmetically- and/or pharmaceutically-acceptable carrier medium.

The stain removing composition may also include ingredients such as stearic acid, sodium lauryl sulfate, EDTA, and the like to aid in desquamation and exfoliation, as well as other conventionally added ingredients. As mentioned, the amount and fineness of the skin-abrading agent will vary depending upon the abrasive effect desired, and will vary among people because of their differing skin types and facial and bodily structures and features.

Additional ingredients can include medications to combat inflammation and/or infection such as, for example, a glucocorticoid and triclosan.

A skin moisturizing composition and/or a photoprotective composition may also be employed in the process of the present invention. The moisturizer and sunscreen are preferably applied after the stain has been removed or lightened from the skin.

The skin moisturizing composition will typically contain at least one skin soothing agent present in an amount sufficient to both soothe previously weakened skin and protect it from further assaults, particularly those from the environment. Suitable skin soothing agents include, but are not limited to, emollients such as silicones, hydrating agents such as polyols, i.e. glycerin and/or propylene glycol which are homogenized together in the presence of an emulsifier, and antioxidants for combating free radicals, such as tocopherol, tocopheryl acetate, ascorbic acid and arginine pyrrolidonecarboxylate.

It is highly recommended that a photoprotective composition containing at least one photoprotective agent be applied onto treated skin in order to protect it from the harmful effects of the sun.

Suitable organic photoprotective agents may be chosen from anthranilates; cinnamic derivatives; dibenzoylmethane derivatives; salicylic derivatives; camphor derivatives; triazine derivatives; benzophenone derivatives; β,β-diphenylacrylate derivatives; benzotriazole derivatives; benzalmalonate derivatives; benzimidazole derivatives; imidazolines; bisbenzoazolyl derivatives; p-aminobenzoic acid (PABA) derivatives; methylenebis(hydroxyphenylbenzotriazole) derivatives; benzoxazole derivatives; screening polymers and screening silicones; dimers derived from α-alkylstyrene; 4,4-diarylbutadienes; and mixtures thereof.

The organic photoprotective agents more particularly preferred may be chosen from the following compounds (CTFA names or chemical names):

Ethylhexyl Salicylate,

Ethylhexyl Methoxycinnamate,

Octocrylene,

Phenylbenzimidazole Sulfonic Acid,

Benzophenone-3,

Benzophenone-4,

Benzophenone-5,

4-Methylbenzylidene Camphor,

Terephthalylidene Dicamphor Sulfonic Acid,

Disodium Phenyl Dibenzimidazole Tetrasulfonate,

2,4,6-Tris(diisobutyl 4′-aminobenzalmalonate)-s-triazine,

Anisotriazine,

Ethylhexyl Triazone,

Diethylhexyl Butamido Triazone,

Methylene Bis-Benzotriazolyl Tetramethyl-butylphenol,

Drometrizole Trisiloxane,

Polysilicone-15,

1,1-Dicarboxy (2,2′-dimethylpropyl)-4,4-diphenylbutadiene,

2,4-Bis[5-1(dimethylpropyl)benzoxazol-2-yl-(4-phenyl)imino]-6-(2-ethylhexyl)imino-1,3,5-triazine, and their mixtures.

Suitable inorganic photoprotective agents may be chosen from pigments or alternatively nanopigments (mean size of the primary particles: generally between 5 nm and 100 nm, preferably between 10 nm and 50 nm) formed of metal oxides which may or may not be coated, such as, for example, nanopigments formed of titanium oxide (amorphous or crystalline in the rutile and/or anatase form), iron oxide, zinc oxide, zirconium oxide or cerium oxide, which are all UV photoprotective agents well known per se. Furthermore, conventional coating agents are alumina and/or aluminum stearate.

The photoprotective agents should be present in the photoprotective composition in an amount sufficient to yield an SPF of at least 15.

The various compositions used in the process of the present invention may be provided in the form of: (i) dispersions of the lotion or gel type, (ii) emulsions with a liquid or semi-liquid consistency of the milk type, obtained by dispersion of a fatty phase in an aqueous phase (O/W) or vice versa (W/o), (iii) suspensions or emulsions with a soft, semi-solid or solid consistency of the cream or gel type, (iv) multiple emulsions (W/O/W or O/W/O), (v) microemulsions, (vi) vesicular dispersions of ionic and/or nonionic type, or (vii) wax/aqueous phase dispersions. These compositions are prepared according to methods known to those of ordinary skill in the art of cosmetics or dermatological formulations.

The oils present in these emulsions can be silicone oils, which may be volatile or nonvolatile, hydrocarbon oils or vegetable oils. These emulsions can additionally comprise non-oily fatty substances, such as shea butter, silicone gums, esters of fatty acids and of fatty alcohols, fatty acids and fatty alcohols.

These compositions can additionally comprise various adjuvants commonly used in the cosmetics field, such as emulsifiers, including glyceryl fatty acid esters, sugar fatty acid esters, sorbitan fatty acid esters, polyethylene glycol fatty acid esters, ethoxylated fatty alcohols and alkylpolyglycosides; fillers, in particular polyacrylamide (Nylon) fibers and/or microbeads, silica, optionally in the form of a colloidal dispersion, and/or organic microspheres which are optionally expanded; preservatives and/or copreservatives, such as caprylyl glycol; colorants; fragrances; pH adjusters, such as neutralizing agents and/or buffering agents; ethanol; and thickening and gelling agents, in particular acrylamide homo- and co-polymers, acrylic homo- and co-polymers, acrylamidomethylpropanesulphonic acid (AMPS) homo- and co-polymers, and xanthan gum.

Of course, a person skilled in the art will take care to choose this or these possible additional compounds and/or their amounts so that the advantageous properties of the compositions according to the invention are not, or not substantially, detrimentally affected by the envisaged addition.

The inventive process involves first massaging a cosmetically suitable stain removing composition containing at least one skin-abrading metal oxide over the stained area of the skin to form a treated skin surface and then removing the stain removing composition to reveal a clean or lightened skin surface.

Additional steps involving the application of a moisturizer and/or a photoprotective composition onto the clean or lightened skin surface may then be employed.

According to another embodiment of the present invention, there is provided a kit containing: (a) a multi-unit receptacle; (b) at least one unit having a sunless tanning composition; (c) at least one unit having a stain removing composition containing at least one skin-abrading metal oxide; (d) optionally, a unit having a skin moisturizing composition; and (e) optionally, a unit having a photoprotective composition.

The present invention will be better understood from the example which follows, which is intended for illustrative purposes only, and is not meant to unduly limit the scope of the invention in any way. Thus, it is intended that the present invention cover the modifications and variations of this invention provided that they come within the scope of the appended claims and their equivalents.

### EXAMPLE

Colorimetric test to determine the amount of color loss from the use of exfoliators on skin treated with a self-tanning product:

A three (3) panel study was conducted with three types of exfoliators, each containing a different skin-abrading agent at the same amount: metal oxide (aluminum oxide or alumina), polyethylene and pumice. Initial color readings were taken five hours after a self-tanner was applied on test sites on the inner volar arms of three panelists. A blind, randomized treatment of the test sites consisting of rinsing (15 seconds), massaging in the exfoliating products (2 minutes), a second rinsing (15 seconds) and pat-drying the sites was conducted. Final color readings were then taken.

| | | | Difference |
|---|---|---|---|
| Exfoliator | Initial Color | Final Color | [ΔE (initial) − ΔE |
| Type | Readings, ΔE | Readings, ΔE | (final)] |
| Alumina | 7.00 | 5.70 | −1.29 |
| Polyethylene | 5.25 | 4.39 | −0.86 |
| Pumice | 5.57 | 4.80 | −0.77 |

The data above shows that the use of the alumina exfoliator resulted in a larger decrease in color compared to the decrease in color resulting from the use of the polyethylene and pumice exfoliators. Therefore, the Alumina exfoliator removed more color from the test sites than the other exfoliators.

What is claimed is:

1. A process for removing or lightening a stain from human skin caused by a sunless tanning product comprising contacting the stain with a stain removing composition containing at least one skin-abrading metal oxide to form a clean or lightened skin surface.

2. The process of claim 1 wherein the at least one skin-abrading metal oxide is chosen from aluminum oxide, magnesium oxide and mixtures thereof.

3. The process of claim 1 further comprising contacting the clean or lightened skin surface with at least one composition chosen from a moisturizing composition and a photoprotective composition.

4. The process of claim 3 wherein the photoprotective composition has an SPF of at least about 15.

5. The process of claim 1 wherein the at least one skin-abrading metal oxide is aluminum oxide.

6. The process of claim 1 wherein the at least one skin-abrading metal oxide is magnesium oxide.

7. The process of claim 1 wherein the stain removing composition is massaged over the stain.

8. A kit comprising:
(a) a multi-unit receptacle;
(b) at least one unit containing a sunless tanning composition;
(c) at least one unit containing a stain removing composition having at least one skin-abrading metal oxide;
(d) optionally, at least one unit containing a moisturizing composition; and
(e) optionally, at least one unit containing a photoprotective composition.

9. The kit of claim 8 wherein the moisturizing and/or photoprotective composition has an SPF of at least about 15.

10. The kit of claim 8 wherein the at least one skin-abrading metal oxide is aluminum oxide.

11. The kit of claim 8 wherein the at least one skin-abrading metal oxide is magnesium oxide.
